(19) United States
(12) Patent Application Publication
(10) Pub. No.: US 2003/0082146 A1
(43) Pub. Date: May 1, 2003

(54) Adenovirus vectors with knobless fibers, and their uses

(76) Inventors: Helmuth H. G. van Es, Haarlem (NL); Victor W. van Beusechem, Amsterdam (NL)

Correspondence Address: TRASK BRITT, P.O. BOX 2550, SALT LAKE CITY, UT, 84110, US

(21) Appl. No.: 10/282,121

(22) Filed: Oct. 28, 2002

Related U.S. Application Data
(63) Continuation of application No. PCT/NL01/00323, filed on Apr. 26, 2001.
(60) Provisional application No. 60/200,160, filed on Apr. 26, 2000.

(51) Int. Cl. A01K 67/00; C12Q 1/70; A61K 48/00; C12N 7/00; C12N 15/861

(52) U.S. Cl. 424/93.2; 800/8; 435/235.1; 435/320.1; 435/5; 435/456

(57) ABSTRACT

Novel adenoviral vectors in which the knob of the fiber protein has been removed and replaced with a binding ligand are provided. Further provided are methods of constructing such vectors and uses therefor. A modified knobless adenovirus comprising a new binding ligand according to the invention has improved capabilities of entry into specific cell types.

### CROSS REFERENCE TO RELATED APPLICATIONS

This application is a continuation of International Application Number PCT/NL01/00323 filed on Apr. 26, 2001, designating the United States of America, corresponding to International Publication Number WO 01/81607 A2, published in English on Nov. 1, 2001, which application itself claims priority from U.S. Provisional Patent Application No. 60/200,160, filed on Apr. 26, 2000. The contents of the entirety of both applications are hereby incorporated by this reference.

### BACKGROUND OF THE INVENTION

1. Field of Invention

The invention relates to the field of molecular genetics and medicine. In particular, the present invention relates to the field of gene therapy, more in particular to gene therapy using adenoviruses. The invention provides novel adenoviral vectors, in which the knob of the fiber has been removed entirely and replaced with a binding ligand, as well as methods of constructing and using such vectors. A modified knobless adenovirus comprising a new binding ligand according to the invention has improved capabilities of entry into specific cell types.

2. State of the Art

Many different methods have been developed to introduce new genetic information into cells. Although many different systems may work on cell lines cultured in vitro, only the group of viral vector mediated gene delivery methods seems to be able to meet the required efficiency of gene transfer in vivo. Thus, for gene therapy purposes, most of the attention is directed toward the development of suitable viral vectors.

Today, most of the attention for the development of suitable viral vectors is directed toward those vectors that are based on adenoviruses. Gene transfer vectors derived from adenoviruses (adenoviral vectors) have a number of features that make them particularly useful for gene transfer:

1) the biology of the adenoviruses is well characterized,

2) the adenovirus is not associated with severe human pathology,

3) the virus is extremely efficient in introducing its DNA into the host cell,

4) the virus can infect a wide variety of cells and has a broad host-range,

5) the virus can be produced at high titers in large quantities,

6) the virus can be rendered replication defective by deletion of the early-region 1 (E1) of the viral genome (Brody and Crystal, 1994), and

7) the virus can be produced free of wild type replicating adenovirus (W097/00326).

Adenoviruses contain a linear double-stranded DNA molecule of approximately 36000 base pairs. It contains identical Inverted Terminal Repeats (ITR) of approximately 90-140 base pairs with the exact length depending on the serotype. The viral origins of replication are within the ITRs exactly at the genome ends. The transcription units are divided in early and late regions. Shortly after infection, the E1A and E1B proteins are expressed and function in transactivation of cellular and adenoviral genes. The early regions E2A and E2B encode proteins (DNA binding protein, pre-terminal protein and polymerase) required for the replication of the adenoviral genome (reviewed in van der Vliet, 1995). The early region E4 encodes several proteins with pleiotropic functions, for example, transactivation of the E2 early promoter, facilitating transport and accumulation of viral mRNAs in the late phase of infection and increasing nuclear stability of major late pre-mRNAs (reviewed in Leppard, 1997). The early region 3 encodes proteins that are involved in modulation of the immune response of the host (Wold et al., 1995). The late region is transcribed from one single promoter (major late promoter) and is activated at the onset of DNA replication. Complex splicing and poly-adenylation mechanisms give rise to more than 12 RNA species coding for core proteins, capsid proteins (penton, hexon, fiber and associated proteins), viral protease and proteins necessary for the assembly of the capsid and shut-down of host protein translation (Imperiale et al., 1995).

The initial step for successful infection is binding of adenovirus to its target cell, a process mediated through fiber protein. The fiber protein has a trimeric structure (Stouten et al., 1992) with different lengths depending on the virus serotype (Signas et al., 1985; Kidd et al., 1993). Different serotypes have polypeptides with structurally similar N and C termini, but different middle stem regions. The first 30 amino acids at the N terminus are involved in anchoring of the fiber to the penton base (Chroboczek et al., 1995), especially the conserved FNPVYP (SEQ ID NO: 1) region in the tail (Amberg et al. 1997). The C-terminus, or knob, is responsible for initial interaction with the cellular adenovirus receptor. After this initial binding, secondary binding between the capsid penton base and cell-surface integrins leads to internalization of viral particles in coated pits and endocytosis (Morgan et al., 1969; Svensson and Persson, 1984; Varga et al., 1991; Greber et al., 1993; Wickham et al., 1993; Hynes, 1992). The array of integrins expressed in cells is complex and will vary between cell types and cellular environment. Although the knob contains some conserved regions, between serotypes, knob proteins show a high degree of variability, indicating that different adenovirus receptors exist.

The interaction of the virus with the host cell has mainly been investigated with the serotype C viruses Ad2 and Ad5. Binding occurs via interaction of the knob region of the protruding fiber with a cellular receptor. The receptor for Ad2 and Ad5, and probably more adenoviruses, is known as the “Coxsackievirus and Adenovirus Receptor” or CAR protein (Bergelson et al., 1997). Internalization is mediated through interaction of the RGD sequence present in the penton base with cellular integrins (Wickham et al., 1993). This may not be true for all serotypes, for example, serotypes 40 and 41 do not contain a RGD sequence in their penton base sequence (Kidd et al., 1993).

At present, six different subgroups of human adenoviruses have been proposed which, in total, encompass approximately 50 distinct adenovirus serotypes. Besides these human adenoviruses, many animal adenoviruses have been identified (see, e.g., Ishibashi and Yasue, 1984).

A serotype is defined on the basis of its immunological distinctiveness as determined by quantitative neutralization with animal antiserum (horse, rabbit). If neutralization shows a certain degree of cross-reaction between two viruses, distinctiveness of serotype is assumed if: A) the hemagglutinins are unrelated, as shown by lack of cross-reaction on hemagglutination-inhibition, or B) substantial biophysical/biochemical differences in DNA exist (Francki et al., 1991). The serotypes identified last (42-49) were isolated for the first time from HIV infected patients (Hierholzer et al., 1988; Schnurr et al., 1993). For reasons not well understood, most of such immunocompromised patients shed adenoviruses that were never isolated from immuno-competent individuals (Hierholzer et al., 1988 and 1992; Khoo et al., 1995).

Besides differences towards the sensitivity against neutralizing antibodies of different adenovirus serotypes, adenoviruses in subgroup C such as Ad2 and Ad5 bind to different receptors as compared to adenoviruses from subgroup B, such as Ad3 and Ad7 (Defer et al., 1990; Gall et al., 1996). Likewise, it was demonstrated that receptor specificity could be altered by exchanging the Ad3 knob protein with the Ad5 knob protein, and vice versa (Krasnykh et al., 1996; Stevenson et al., 1995 and 1997). Serotypes 2, 4, 5 and 7 all have a natural affiliation towards lung epithelia and other respiratory tissues. In contrast, serotypes 40 and 41 have a natural affiliation towards the gastrointestinal tract. These serotypes differ in at least capsid proteins (penton-base, hexon), proteins responsible for cell binding (fiber protein), and proteins involved in adenovirus replication. It is unknown to what extend the capsid proteins determine the differences in tropism found between the serotypes. It may very well be that post-infection mechanisms determine cell type specificity of adenoviruses. It has been shown that adenoviruses from serotypes A (Ad12 and Ad31), C (Ad2 and Ad5), D (Ad9 and Ad15), E (Ad4) and F (Ad41) all are able to bind labeled, soluble CAR (sCAR) protein when immobilized on nitrocellulose.

Furthermore, binding of adenoviruses from these serotypes to Ramos cells, that express high levels of CAR but lack integrins (Roelvink et al., 1996), could be efficiently blocked by addition of sCAR to viruses prior to infection (Roelvink et al., 1998). However, the fact that (at least some) members of these subgroups are able to bind CAR does not exclude that these viruses have different infection efficiencies in various cell types. For example, subgroup D serotypes have relatively short fiber shafts compared to subgroup A and C viruses. It has been postulated that the tropism of subgroup D viruses is to a large extent determined by the penton base binding to integrins (Roelvink et al., 1996; Roelvink et al., 1998). Another example is provided by Zabner et al. (1998) who have tested 14 different serotypes on infection of human ciliated airway epithelia (ACAE@) and found that serotype 17 (subgroup D) was bound and internalized more efficiently then all other viruses, including other members of subgroup D. Similar experiments using serotypes from subgroup A-F in primary fetal rat cells showed that adenoviruses from subgroups A and B were inefficient whereas viruses from subgroup D were most efficient (Law et al., 1998). Also in this case, viruses within one subgroup displayed different efficiencies. The importance of fiber binding for the improved infection of Ad17 in CAE was shown by Armentano et al. (WO 98/22609) who made a recombinant LacZ Ad2 virus with a fiber gene from Ad17 and showed that the chimeric virus infected CAE more efficiently than LacZ Ad2 viruses with Ad2 fibers.

Thus, despite their shared ability to bind CAR, differences in the length of the fiber, knob sequence and other capsid proteins, e.g., penton base, of the different serotypes may determine the efficiency by which an adenovirus infects a certain target cell. Of interest in this respect is the ability of Ad5 and Ad2 fibers, but not of Ad3 fibers, to bind to fibronectin III and MHC class 1-derived peptides. This suggests that adenoviruses are able to use cellular receptors other than CAR (Hong et al., 1997).

Serotypes 40 and 41 (subgroup F) are known to carry two fiber proteins differing in the length of the shaft. The long shafted 41L, fiber is shown to bind CAR, whereas the short shafted 41 S is not capable of binding CAR (Roelvink et al., 1998). The receptor for the short fiber is not known.

Adenoviral vectors are also ideally suited for applications in functional genomics (see WO 99/64582). The vectors can be used to build gene expression libraries which can be used with specific cell based assays to search for genes or antagonists of those genes that give a desired phenotype. They can also be used to validate further genes that have been isolated using other gene selection techniques, such as comparative expression profiling and subtraction techniques. Validation using adenoviral vectors can be done in vitro as well as in vivo using either in situ or in vitro cell or tissue based assays, or appropriate animal models.

Most adenoviral gene delivery vectors currently used in functional genomics, gene therapy, or vaccination are derived from the serotype C adenoviruses Ad2 or Ad5. The vectors have at least a deletion in the El region, where novel genetic information can be introduced. The E1 deletion renders the recombinant virus replication defective. It has been demonstrated extensively that recombinant adenovirus, in particular serotype 5, is suitable for efficient transfer of genes in vivo to the liver, the airway epithelium and solid tumors in animal models and human xenografts in immuno-deficient mice (Bout, 1996 and 1997; Blaese et al., 1995).

The use of adenoviral vectors in functional genomics includes building gene expression libraries and in vitro and in vivo gene validation with appropriate meaningful cell-based assays or animal models for a particular human disease. Transfer and subsequent expression of a cDNA into a desired cell type may lead in relevant phenotypic changes that may or may not confirm the role a particular cDNA plays in a particular disease. Alternatively, such an exercise may lead to better insight into the validity of using a particular cDNA as a target for therapeutic intervention. In addition to sense copies of a gene or genes under investigation, antisense copies may be cloned into the adenoviral vector and used for validation studies.

As described herein, recombinant adenoviral vectors are being used as gene delivery vectors in a variety of gene therapy strategies and in functional genomics approaches. The major advantage of adenoviral vectors over other vector systems is their unparalleled efficiency of in vivo gene transfer into cells of various organs.

However, while the efficient transduction of many different human tissues is, on the one hand, an important attribute of adenoviral vectors, this promiscuous tropism represents, on the other hand, a limiting feature for their use in gene therapy. In vivo delivery of adenoviral vectors results not only in the transduction of desired target cells, but also in unwanted gene transfer into cells that are not a target for the therapy, most notably liver cells (Herz and Gerard, 1993; Huard et al., 1995). Due to this vector sequestration by non-target cells, much higher vector doses are needed. These higher doses impose an increased risk for unwanted side-effects of the procedure, for example, by direct toxicity or host immune responses against the vector. Moreover, in anti-cancer gene therapy approaches based on expression of genes encoding cytotoxic or immune-modulating products, transduction of non-cancerous cells may result in harmful side-effects of the therapy.

Additionally, some cell types or tissue types are poorly transduced using the current adenoviral vectors. For instance, endothelial cells, smooth muscle cells, many types of tumor cells and T lymphocytes are not easily transduced by the current generation of adenovirus vectors. For many gene therapy or functional genomics applications, preferably these types of cells should be genetically modified. Disease areas for which efficient gene transfer into these cell types is desirable include, but are not limited to, autoimmune disorders, cancer, infectious diseases, cardiovascular diseases and bone disorders. In conclusion, the characteristics of the current adenoviral vectors limit their use in specific applications.

Furthermore, the vectors are not ideally suited for delivering additional genetic material to organs other than the liver. The liver can be particularly well transduced with vectors derived from Ad2 or Ad5. Delivery of such vectors via the bloodstream leads to a significant delivery of the vectors to the cells of the liver. In therapies where other cell types than liver cells need to be transduced, some means of liver exclusion must be applied in order to prevent uptake of the vector by these cells. Current methods rely on the physical separation of the vector from the liver cells, most of these methods rely on localizing the vector and/or the target organ via surgery, balloon angioplasty, or direct injection into an organ via, for instance, needles. Liver exclusion is also being practiced by surgical targeting by delivery of the vector to compartments in the body that are essentially isolated from the bloodstream. This targeting prevents, or at least reduces, transport of the vector to the liver.

Although these methods mostly succeed in avoiding gross delivery of the vector to the liver, most of the methods are crude and still have considerable leakage and/or have poor target tissue penetration characteristics. In some cases, inadvertent delivery of the vector to liver cells can be toxic to the patient. For instance, delivery of a herpes simplex virus (HSV) thymidine kinase (TK) gene for the subsequent killing of dividing cancer cells through administration of ganciclovir is quite dangerous when also a significant amount of liver cells are transduced by the vector. Significant delivery and subsequent expression of the HSV-TK gene to liver cells is associated with severe toxicity. Thus, there is a clear need for an inherently safe vector provided with the property of a reduced transduction efficiency of liver cells. This safety requirement is even more eminent in the context of replication-competent or conditionally-replicating adenoviruses. There is an increasing interest in the field of gene therapy to employ the lytic activity of such viruses for the eradication of cancerous, pre-malignant or other hyper-proliferating cells such as, for instance, arthritic synovial cells, from the human body. It will be clear that in this application, restriction of the infection to the target cells is crucial. Thus, there also exists a clear need for targeted replication-competent or conditionally-replicating adenoviruses.

In vitro or ex vivo gene transfer for functional genomics using currently available adenoviral vectors is also limited for certain cell types, in particular, cells of the hemopoietic system, as well as cells of the vasculature such as endothelial cells. The characteristics of the current adenoviral vectors limit their use in specific applications. Bispecific antibodies consisting of an antibody against adenoviral knob and an antibody against the pantropic marker CD3 have been used to transfer genes into T lymphocytes. The production of bispecific antibodies is done using chemical coupling methods such as succinimidyl-3-(2-pyridyldithiol)-propionate (SPDP) as a cross linking agent. Even though coupling of antibodies is technically feasible, these methods are prone to be difficult in terms of reproducibility. Furthermore, every time a transduction is done, the adenovirus needs to be preincubated with the bispecific antibodies to generate the targeted adenovirus creating another variable.

Therefore, adenoviral vector systems could potentially be much improved if specific gene transfer into only the desired target cells could be accomplished. There exists a clear need for the development of truly targeted adenovirus vectors. This targeting requires two elements: the complete ablation of native adenovirus tropism and the introduction of a novel binding affinity. For pharmaceutical production of such targeted adenovirus vectors, it is much preferred that such vectors are produced as single-reagent genetic medicines. In addition, for application of targeted replication-competent or conditionally-replicating adenoviruses, it is essential that the novel binding affinity is retained in the virus progeny. Therefore, also in this context, it is preferred that such targeted replication-competent or conditionally-replicating adenovirus is produced as a single-component reagent. It will be clear from the above that it is much preferred that the novel binding affinity is incorporated as an integral component of the adenovirus particle, rather than being provided as part of a second component, such as a bispecific antibody.

### BRIEF SUMMARY OF THE INVENTION

The current invention provides novel adenoviral vectors that are truly targeted to specific cell types. This targeting is obtained by the complete ablation of the native adenovirus tropism and the introduction of a novel binding affinity. Means and methods are disclosed herein for the construction of such novel adenoviral vectors, and their uses.

In the present invention, adenovirus fiber proteins are provided in which the knob domain is deleted and replaced with other distinct protein moieties. The first moiety is an alpha-helix domain that serves to provide a trimerization function for the knobless fiber protein and the second moiety mediates specific binding to the target cell. Adenoviral vectors are provided that carry one of the knobless fiber proteins TSC and TSFLC, each comprising an alpha-helix domain from Moloney Murine Leukemia Virus envelope glycoprotein and only differing in the linker peptide between the fiber shaft and alpha-helix domains. Two carboxy-terminal mimic targeting ligands were coupled to TSC and TSFLC via a flexible linker peptide, i.e., a Myc-epitope and a 6His-tag. The targeted knobless fiber molecules were properly expressed and imported into the nucleus of adenovirus packaging cells. TSFLC molecules were incorporated as functional trimers into the adenovirus capsid. Its carboxy-terminal ligands were functionally exposed on the surface of the intact virion for specific binding to their target molecules. Moreover, the TSFLC knobless fiber protein mediated targeted gene delivery into cells displaying artificial receptors for the carboxy-terminal 6His ligand. Hence, TSFLC knobless fiber molecules are prototype substrates for versatile addition of targeting ligands to generate truly targeted adenoviruses.

The present invention provides adenoviral vectors comprising at least one normative amino acid sequence, wherein the normative amino acid sequence replaces the knob domain of the adenovirus fiber protein and provides the adenoviral vector with desired cell type specificity. Normative means not derived from adenoviral protein structures or sequences. Adenoviral vector means an adenoviral particle, or a part thereof, which is capable of at least interacting with a cell. In a preferred embodiment, the invention provides adenoviral vectors, wherein the normative amino acid sequence that replaces the adenovirus fiber knob domain is a binding ligand that enables binding to a certain specific cell type. Binding ligand means anything that binds or interacts with proteinaceous or non-proteinaceous compounds exposed on the surface of a cell. Examples of such ligands are hormones or fragments thereof, monoclonal antibodies or fragments thereof, heavy chains, light chains, single chain Fv fragments, Fab fragments, short circulating peptides, or other non-proteinaceous substances such as small molecules. In a more preferred embodiment, the invention provides adenoviral vectors, wherein the binding ligand that enables binding to a certain specific cell type is coupled to the adenoviral vector via a flexible linker peptide. Furthermore, the invention provides adenoviral vectors, wherein the binding ligand for binding to a specific cell type comprises a trimerization domain. In a preferred embodiment, the trimerization domain is derived from a viral membrane fusion protein, such as a retroviral envelope glycoprotein. In a more preferred embodiment, the trimerization domain is derived from a Moloney Murine Leukemia Virus or from a Rous Sarcoma Virus.

In another aspect, the invention provides adenoviral vectors, wherein the binding ligand for binding to a specific cell type comprises a (carboxy-terminal) Myc-epitope or a derivative thereof. In a more preferred aspect of the invention, the binding ligand for binding to a specific cell type comprises, next to the Myc-epitope or a derivative thereof, a 6his-peptide comprising 6 Histidine residues.

The present invention also provides adenoviral vectors, wherein the binding ligand for binding to a specific cell type that replaces the knob domain of the fiber protein on the adenoviral vector and which comprises normative amino acid sequences is derived from a monoclonal antibody directed against the extracellular domain of a cationic amino acid transporter protein, such as hCAT1. In another aspect of the invention, the binding ligand for binding to a specific cell type present on said adenoviral particle is derived from a monoclonal antibody against an epithelial cell adhesion molecule, such as the 17-1A antigen. In a preferred embodiment, the invention provides adenoviral vectors, wherein the normative amino acid comprises more than 25 amino acids.

The present invention also provides methods of improving the recognition by adenoviral vector for a specific cell, the method comprising contacting the specific cell with the adenoviral vector. Contacting means incubating the specific cell with the adenoviral vector under culturing conditions that allow an interaction of the adenoviral vector with a compound on the surface of the cell, with a possible subsequent entry of the adenoviral vector and/or its nucleic acid content in the specific cell.

In another embodiment, the invention provides a method of producing a library of adenoviral vectors with desired cell type specificity for use in functional genomic applications, the method comprising the step of assembling the adenoviral vectors in a cell capable of doing so, in which the normative amino acid sequence encoded by a nucleic acid is expressed in the cell. The invention further provides cells, tissue containing such cells and non-human animals containing such tissue and cells, infected with said adenoviral vectors.

### BRIEF DESCRIPTION OF THE DRAWINGS

The foregoing features and advantages of the invention will be apparent from the following description and apparent from the accompanying drawings.

FIG. 1 is an illustration of the TSCmychis and TSFLCmychis genes.

FIG. 2A illustrates Myc-epitope containing proteins as detected by immunocytochemistry allowing analysis of the intracellular localization of the chimeric proteins.

FIG. 2B illustrates that the control protein LacZmychis is detected predominantly in the cytoplasm of transfected cells.

FIG. 2C illustrates that the protein TSCmychis is accumulated in the cell nuclei.

FIG. 2D illustrates that the protein TSFLCmychis is accumulated in the cell nuclei.

FIG. 3A is a Western analysis of oligomeric structures of knobless fiber molecules under semi-native versus denaturing conditions using an anti-Myc antibody.

FIG. 3B illustrates the binding of TSCmychis and TSFLCmychis proteins to Ni-NTA molecules.

FIG. 4 is a Western analysis for wild-type and knobless fiber variants showing detection of wild-type fiber trimers in AdGFP, ADGFP-TSCmychis and AdGFP-TSFLCmychis. No knobless fibers were detected on AdGFP particles.

FIG. 5A illustrates recovery of functional GFP-vector from the Ni-NTA beads at different stringencies of competition with imidazol.

FIG. 5B is a FACS analysis of 293.HissFv.rec cells for GFP-expression.

FIG. 6 shows that when the CAR-binding site on the wild-type fiber is blocked, gene transfer by AdGFP drops approximately 25-fold in the first experiment and 30-5-fold in the second experiment. AdGFP-TSFLCmychis virus, however, still exhibits more than 10% gene transfer in the presence of the anti-fiber knob antibody.

### DETAILED DESCRIPTION OF THE INVENTION

Disclosed are means and methods to develop truly targeted adenovirus vectors by deletion of the fiber knob comprising the receptor-binding site and its replacement with two distinct protein moieties. The knob deletion results in significant ablation of native receptor binding affinity. The first added protein moiety serves to provide a relatively simple structure that complements for the trimerization function of the knob and the second moiety should mediate specific binding to the target cell. The trimerization- and targeting-ligand moieties are coupled via a flexible linker peptide, such that they do not perturb each other's folding. This facilitates the use of a large variety of targeting ligands. In selecting a useful trimerization domain for the knobless fiber, we reasoned that it should be functional in the cytoplasm where fiber trimers are formed (Hong and Engler, 1991), and should remain stable in the nuclear environment where adenovirus capsids are assembled, as well as in the extracellular environment where the novel targeting molecule mediates infection of the target cell. The remaining domains of the fiber, i.e., the tail and the shaft, are completely retained in the chimeric molecule to provide nuclear import, anchoring to the viral capsid and prevention of fiber-independent infection (Hong and Engler, 1991; Roelvink et al., 1996).

A common motif providing peptide multimerization in many proteins is a short coiled-coil consisting of amphipatic a-helices displaying a characteristic pattern of heptad repeats containing hydrophobic residues at the first and fourth positions (Cohen and Parry, 1990). Depending on the distribution of leucine residues and the a-branched amino acids valine and isoleucine over the first and fourth positions, the helices form dimers, trimers, or tetramers (Harbury et al., 1993 and 1994). Trimeric coiled-coils are almost exclusively found in secreted proteins and in the extracellular moiety of transmembrane proteins. They are often found adjacent to the fusion peptides of viral membrane fusion proteins, including influenza hemagglutinin, c-type retrovirus envelope glycoprotein and coronavirus spike protein, where they have been proposed to play a role in membrane fusion events following fusion activation (Chambers et al., 1990; Bullough et al., 1994; Wild et al., 1994; Fass et al., 1996; Chan et al., 1997).

In the present invention, we linked the alpha-helix domain from the transmembrane subunit p15E of Moloney Murine Leukemia Virus (MoMLV) envelope glycoprotein (Fass et al., 1996) to the adenovirus fiber tail and shaft. A trimeric coiled-coil motif can substitute for the fiber knob trimerization function in a knobless fiber mutant.

Two model knobless fiber proteins with a carboxy-terminal Myc/6His-peptide epitope were expressed in adenovirus vectors, one of which exhibited all functions required for its use in targeted gene delivery. It was properly incorporated into the adenovirus capsid where its carboxy-terminal 6His- and Myc-epitopes were accessible for specific binding to nickel ions and immobilized anti-Myc antibodies, respectively. Moreover, these knobless fibers mediated targeted gene transfer into cells displaying anti-His scFv artificial receptors. Hence, the novel knobless fiber molecules described herein are prototype substrates for addition of targeting ligands to generate truly targeted adenoviruses.

Construction of Knobless Adenovirus Fiber Genes

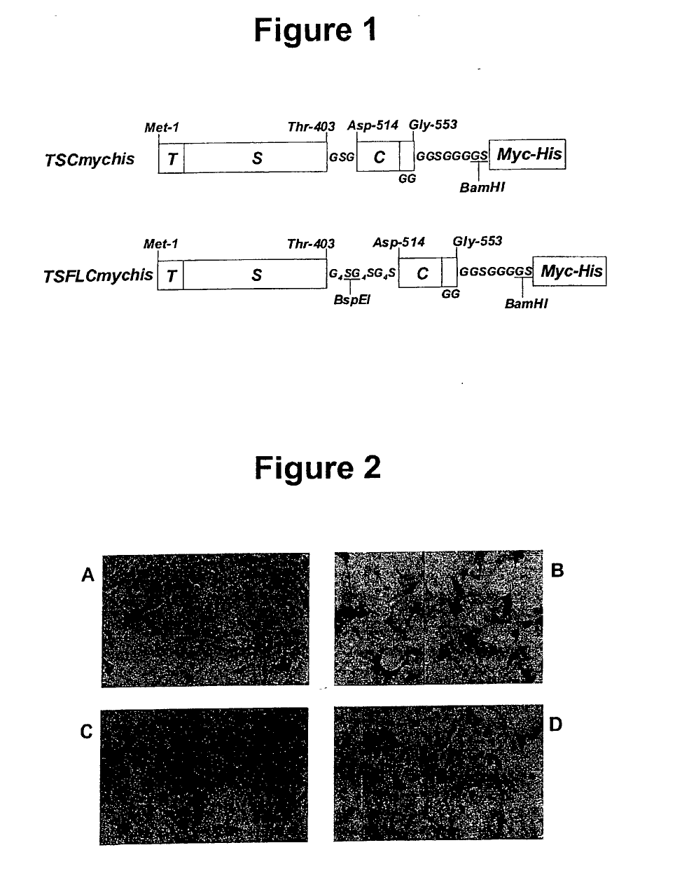

Adenovirus vectors were constructed that carry knobless fiber variants on their capsid. This was done by replacing the fiber knob with an alternative trimerization domain. Two chimeric genes encoding the entire Ad5 fiber tail and shaft domains and a trimerization domain derived from the MoMLV envelope glycoprotein were made. The new genes were constructed using PCR techniques and were designated TSC and TSFLC, respectively (FIG. 1). In both molecules, Ad5 fiber sequences are included that encode Met-1 through Thr-403, where Thr-403 is the last residue of the highly conserved TLWT (SEQ ID NO:2) motif that delineates the start of the fiber knob. The trimerization domain in both genes consists of the MoMLV envelope glycoprotein region from Asp-514 through Gly-553. This sequence covers the 33-residue trimeric coiled-coil from Asp-515 to Leu-547 (Fass et al., 1996). In TSC, the fiber shaft and p15E helix domains are separated by the sequence Gly-Ser-Gly, in TSFLC these domains are linked via a classical (Gly₄Ser)₃ (SEQ ID NO:3) linker commonly used in single chain antibodies. Thus, TSC and TSFLC represent fusion proteins with a linkage between the fiber-shaft and alpha-helix domains that allows minimal or maximal folding freedom, respectively. TSC and TSFLC each have a carboxy-terminal (Gly₄Ser)₂ (SEQ ID NO:4) flexible linker extension with a unique BamH1 restriction site to allow targeting ligand addition. The flexible linker serves to allow both the trimerization domain and the targeting-ligand to adopt their functional conformations.

We constructed derivatives of TSC and TSFLC with carboxy-terminal Myc-epitope and 6-His tag extensions for the purpose of detection and to serve as a mimic targeting ligand. These molecules were designated TSCmychis and TSFLCmychis, respectively (FIG. 1). The carboxy-terminal tags in TSCmychis and TSFLCmychis were coupled via the BamHI site in the flexible linker and are thus presented in the same fashion as cell type-specific targeting ligands would be presented by the knobless fibers. Hence, TSCmychis and TSFLCmychis could serve as model molecules to test TSC and TSFLC knobless fibers for properties relevant to their use in genetically targeted adenovirus vectors. These properties include expression in adenovirus packaging cells, trimerization, nuclear import, stable capsid incorporation, presentation for specific binding of the carboxy-terminal tags on the intact virion, and functional knobless fiber-mediated infection of cells expressing artificial receptors for His-tagged adenoviruses.

Expression and Nuclear Import of Knobless Adenovirus Fibers in Mammalian Cells.

Expression of TSCmychis and TSFLCmychis knobless fiber proteins was initially evaluated by transient transfection of eukaryotic expression plasmids in 911 packaging cells (Fallaux et al., 1996). This allows the analysis of nuclear import in E1-complementing cells in the absence of a cytopathic effect. The transcription of the chimeric genes was driven by the CMV immediate early promoter and the expression was augmented by inserting the adenovirus tripartite leader (TPL) sequence between the CMV promoter and knobless fiber coding region. As a control, a vector expressing the bacterial beta-galactosidase (LacZ) gene with C-terminal Myc/6His-peptide was used. Twenty-four hours after transfection of these constructs into 911 cells, Myc-epitope containing proteins could be detected by immunocytochemistry allowing analysis of the intracellular localization of the chimeric proteins (see FIG. 2A). As was expected, the control protein LacZmychis was detected predominantly in the cytoplasm of transfected cells (FIG. 2B). In contrast, TSCmychis (FIG. 2C) and TSFLCmychis (FIG. 2D) proteins accumulated in the cell nuclei, where adenovirus capsids assemble. Thus, the nuclear localization signal in the Ad fiber tail domain is functionally intact and correctly targets the knobless fiber molecules with their carboxy-terminal peptide-ligand to the cell nucleus. An observation that the amino-terminal 10 amino acids of the adenovirus type 2 fiber suffice to dominantly translocate the cytoplasmic beta-galactosidase protein to the nucleus (Hong and Engler, 1991) suggest that other ligands also allow nuclear import.

Trimerization of Knobless Adenovirus Fiber Protein and Accessibility of the C-Terminal Tag for Specific Binding.

To produce knobless fiber expressing adenovirus vectors on conventional packaging cells, we cloned the TSCmychis and TSFLCmychis genes downstream from an Ad Major Late Promoter (MLP) and TPL in the El-region of an E1/E3-deleted adenovirus vector carrying an expression cassette for Enhanced Green Fluorescent Protein (EGFP). The resulting vectors AdGFP-TSCmychis and AdGFP-TSFLCmychis, co-express the knobless fiber variants with wild-type fibers to allow propagation on 293 packaging cells. As a negative control for knobless fiber expression, AdGFP virus with an EGFP expression cassette as the only E1-insert was produced. All three viruses were expanded on 293 cells to yield high-titer virus stocks.

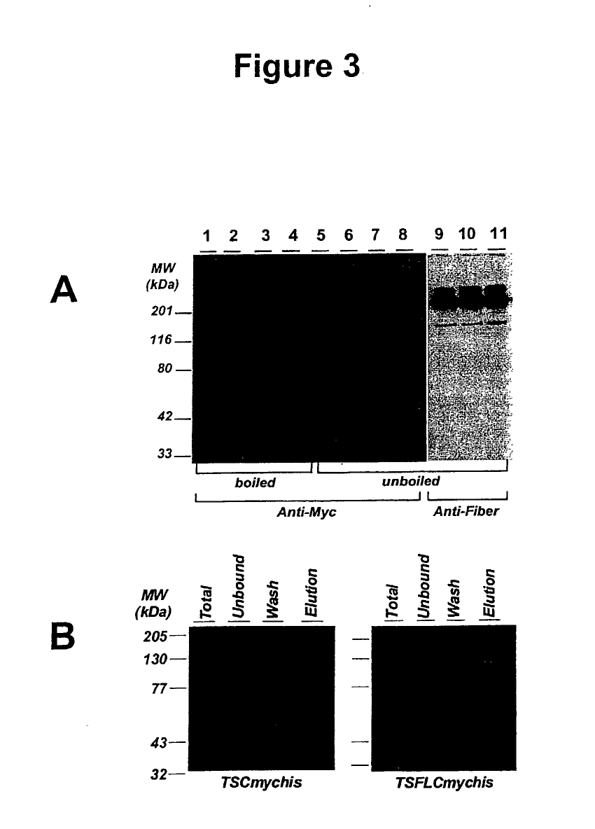

To analyze the knobless fiber proteins produced by the recombinant Ad vectors, 293 cells were infected at high MOI, resulting in 100% EGFP expressing cells the next day, and after 48 hours complete CPE was evident. The oligomeric structure of the knobless fiber molecules was assessed by Western analysis under semi-native versus denaturing conditions using an anti-Myc antibody (FIG. 3A). As can be seen, both TSCmychis and TSFLCmychis were expressed by Ad infected 293 cells and exhibited the expected molecular weight of approximately 50 kDa. Under semi-native conditions, both molecules were found as monomers and multimers. The apparent molecular weight of the knobless fiber multimers was larger than expected for homotrimers (more than 200 kDa observed versus 150 kDa expected). However, analysis of the same samples with an antibody recognizing the trimeric wild-type fiber showed that this protein also migrated at an apparent molecular weight larger than 200 kDa, where approximately 180 kDa was expected (FIG. 3A). This is a well-described phenomenon that can be explained by partial unfolding of the fiber tail and shaft under laboratory conditions (Mitraki et al., 1999). As can be seen in FIG. 3A, only an estimated 5-10% of the knobless fiber molecules were detected as multimers, which is much less efficient than wild-type fiber trimerization (Hong and Engler, 1996). In different independent experiments performed, the efficiency of trimerization detected on semi-native gels varied considerably and was sometimes even undetectable. We attribute this to a rather weak stability of the molecules under laboratory conditions. This was not unexpected, because p5E coiled-coils have a low thermostability (Fass and Kim, 1995).

We next investigated if the C-terminal 6His-tag that was coupled to the knobless fiber molecules via a flexible linker peptide was accessible for binding. To this end, cell lysates of infected 293 cells were incubated with nickel-nitrilotriacetic acid (Ni-NTA) metal-affinity matrices. Unbound material was washed away vigorously and specifically bound 6His-containing proteins were eluted by competition with imidazol. Individual fractions were subjected to Western analysis. As can be seen in FIG. 3B, TSCmychis and TSFLCmychis proteins bound to Ni-NTA matrices, confirming that the C-terminal ligands attached to both knobless fiber variants are accessible for binding.

Incorporation of Knobless Fiber Molecules in Adenovirus Capsids.

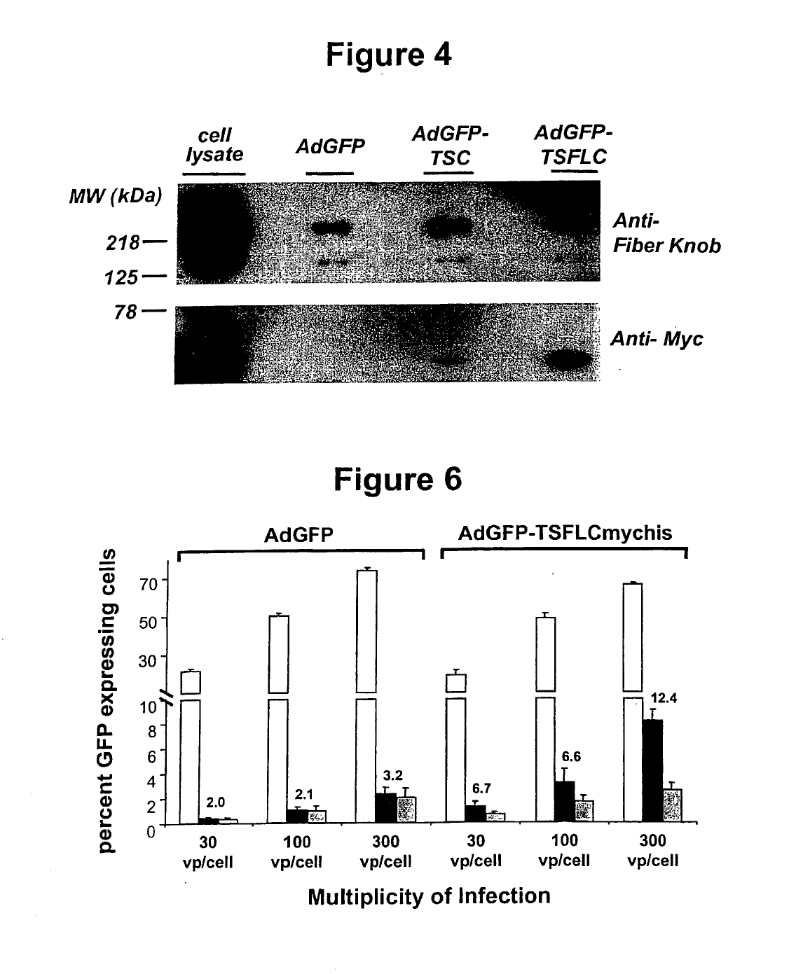

To investigate if the knobless fiber molecules that are produced in packaging cells during recombinant adenovirus replication are incorporated into complete adenovirus capsids, high-titer virus stocks of vectors AdGFP, AdGFP-TSCmychis, and AdGFP-TSFLCmychis were prepared. Complete adenovirus particles were purified by CsCl banding and subjected to Western analysis for wild-type and knobless fiber variants. As can be seen in FIG. 4, wild-type fiber trimers were detected in all three samples. As expected, knobless fibers were not detected on AdGFP particles. In contrast, the knobless fiber mutant TSFLCmychis was clearly present in the CsCl-purified AdGFP-TSFLCmychis batch. TSCmychis molecules were also co-purified with intact Ad particles, but to a much lesser extent. Thus, at least TSFLCmychis chimeric fibers were efficiently co-purified with Ad particles on CsCl density gradients. This finding also provides further proof for trimerization of TSFLCmychis molecules, because only fiber trimers can bind to the viral capsid (Novelli and Boulanger, 1991; Santis et al., 1999).

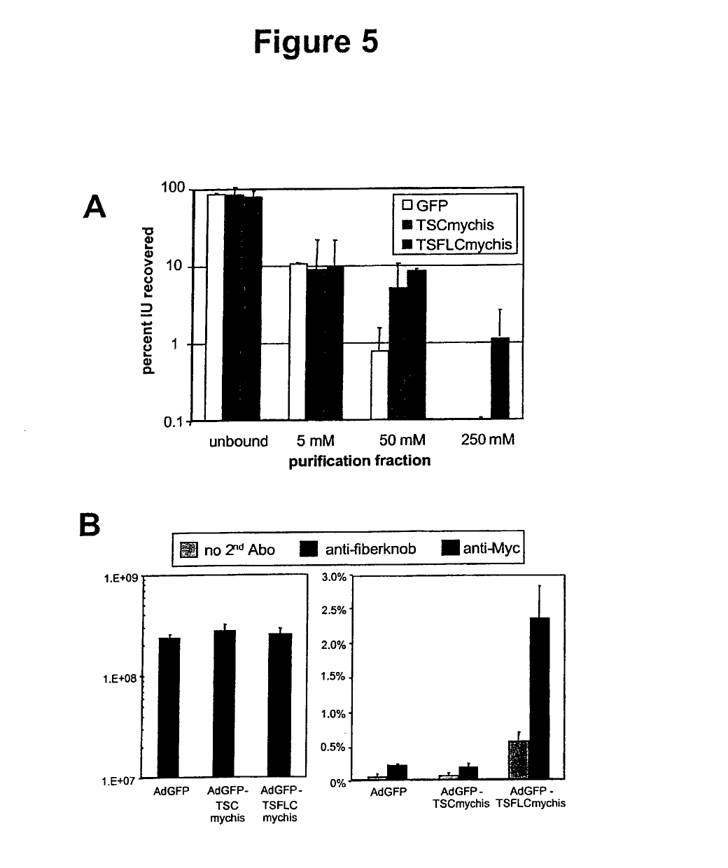

To further corroborate the capsid incorporation, we investigated if functional virus particles could be bound with specificity to mimic receptors for the carboxy-terminal epitopes that are exposed on the knobless fiber molecules. Functional exposure of the 6His-tag on the virus capsid was tested by binding to Ni-NTA beads. Approximately 10¹² particles of each of the three virus stocks were incubated with Ni-NTA beads and unbound material, as well as material eluted from the beads at low, intermediate, or high stringency was collected. Recovery of virus particles in the different fractions was determined in two different manners. First, we isolated viral DNA from the particles in each fraction and visualized it on an agarose gel. Viral DNA was detected in the total, unbound, and low and intermediate stringency elution fractions of all three viruses. In the high-stringency elution fraction, however, viral DNA was only found if AdGFP-TSFLCmychis particles had been incubated with the beads. This indicated specific high-affinity binding of complete Ad.GFP-TSFLCmychis particles containing full-length viral DNA to the Ni-NTA beads. Second, we quantified the binding to Ni-NTA by measuring the functional GFP-vector titers of the different Ni-NTA fractions on 293.HissFv.rec cells (Douglas et al., 1999). The recovery of functional GFP-vector from the Ni-NTA beads at different stringencies of competition with imidazol is shown in FIG. 5A. The negative control virus AdGFP exhibited an elution pattern that was very similar to the predicted distribution of unbound virus over the different fractions, due to the residual volume of the pellet of Ni-NTA beads after each incubation step. Thus, as expected, wild-type fiber-expressing AdGFP did not bind to Ni-NTA. The AdGFP-TSCmychis virus showed an increased recovery in the intermediate stringency elution fraction, but no sign of high-affinity virus binding to Ni-NTA. In contrast, the 50 mM and 250 mM imidazol fractions of AdGFP-TSFLCmychis contained approximately 9.5 and 1.3% of the total recovered virus, respectively. This is much more than may be explained by non-bound virus (predicted aspecific recoveries: 0.5% and 0.02%, respectively). Hence, intact infectious AdGFP-TSFLCmychis particles bound to Ni-NTA with high affinity via their 6His-tagged knobless fibers.

We also tested functional exposure of the Myc-tag at the carboxy-terminus of the knobless fibers on the virus capsid. To this end, plastic culture plates were coated with anti-Myc antibody 9E 10 and serial dilutions of CsC 1-gradient purified virus were allowed to bind. As a positive control, the plates were coated with anti-fiber knob antibody 1D6.14. After extensive washing, 293.HissFv.rec cells were seeded into the wells and cultured overnight. The next day, the cells were harvested and analyzed for GFP-expression on a FACS (FIG. 5B). Anti-fiber knob coated plates very efficiently bound all three viruses, i.e., 24-28% of the virus particles were bound and subsequently infected the 293.HissFv.rec cells resulting in GFP expression the next day. Anti-Myc antibody coated plates showed a very low background binding of the negative control AdGFP virus (0.2%). AdGFP-TSCmychis exhibited a binding pattern similar to that of AdGFP. Hence, the Myc-tag was not functionally exposed on the AdGFP-TSCmychis particles. In contrast, approximately 2.3% functional AdGFP-TSFLCmychis viruses bound to the anti-Myc antibody, with only 0.6% of these viruses adhering to the control plates. This finding confirms that on the intact AdGFP-TSFLCmychis capsid, in addition to the 6His-tag, also the Myc-epitope is presented for binding.

Taken together, several independent lines of evidence show that TSFLCmychis knobless fibers are incorporated in complete and functional adenovirus vectors and functionally expose both carboxy-terminal tags for specific binding on 1-2% of the viral particles. Capsid incorporation of TSCmychis molecules was much less efficient, suggesting that flexibility of the linkage between the fiber shaft and trimerization domain is important. This was a surprising observation, because trimerization and nuclear import efficiencies of the two variants seemed comparable. In addition, recent knowledge on the structure of the fiber shaft suggests that residues 393-398 that were included near to the linkage-site in both knobless fiber variants can form a flexible linker (Van Raaij et al., 1999).

Targeted gene transfer by knobless fiber-carrying adenoviruses into cells displaying an artificial receptor.

Knobless fiber-mediated gene transfer was demonstrated using 293.HisscFv.rec cells (Douglas et al., 1999). 293.HisscFv.rec cells display an anti-His single-chain antibody (scfv) variant on their surface that functions as an artificial receptor for adenoviruses carrying 6His-tagged fibers. Hence, these cells can be used to test the ability of His-tagged knobless fibers to function as primary binding-molecules for CAR-independent adenovirus-mediated gene transfer. 293.HissFv.rec cells were infected with AdGFP or AdGFP-TSFLCmychis vectors at various MOI and EGFP expression was measured the next day. To discriminate between wild-type and targeted infection, the fiber-to-receptor interactions were blocked with anti-fiber knob and anti-Myc antibodies, respectively. FIG. 6 shows that when the CAR-binding site on the wild-type fiber was blocked, gene transfer by AdGFP dropped approximately 25-fold in the first experiment and 30-50 fold in the second experiment. AdGFP-TSFLCmychis virus, however, still exhibited more than 10% gene transfer in the presence of the anti-fiber knob antibody. This shows that AdGFP-TSFLCmychis virus can infect cells via a CAR-independent pathway. Addition of an anti-Myc antibody that blocks the carboxy-terminus of the knobless fiber reduced this gene delivery to a level similar to that of the blocked AdGFP control. Thus, AdGFP-TSFLCmychis vectors employed their knobless fibers for targeted gene transfer via the artificial receptor displayed on the 293.HisscFv.rec cell surface. Since the virus binding studies had indicated that AdGFP-TSFLCmychis particles carried only approximately 1-2% TSFLCmychis molecules compared to the amount wild-type fibers, we may conclude that TSFLCmychis knobless fiber-mediated gene delivery is very efficient.

In a preferred embodiment of the present invention, in the targeted adenoviruses, the wild-type fiber gene is replaced in its entirety by targeted TSFLC derivatives. For this purpose, it is of note that both carboxy-terminal peptide tags were accessible for functional binding. Preferably, a cell type-specific binding ligand is incorporated in place of the Myc-epitope, while the His-tag is employed for vector propagation on 293.HissFv.rec cells.

The invention provides a universal method to construct targeted knobless fibers that incorporate one or more of a variety of ligands with cell type-specific binding affinity at their carboxy-terminus. The DNA sequence encoding a ligand of choice is obtained using methods known in the art including, but not limited to, synthetic oligonucleotide synthesis, polymerase chain reaction, enzymatic modification of a DNA sequence comprising the DNA sequence encoding a ligand of choice, and the like. The ligand of choice is a peptide with a certain binding specificity that distinguishes target cells from other (non-target) cells. The ability to distinguish target cells from non-target cells may lie in the abundance of a certain target molecule on the surface of a certain subset of cells. In one variation of the invention, the ligand peptide is selected on the basis of knowledge on the interaction of a certain target molecule with a natural ligand for the target molecule. For many cell type-specific antigens, protein ligands have been identified, such as, e.g., cytokines binding to their cellular receptor or cell adhesion molecules binding to an adhesion counterpart on a different cell. Alternatively, the peptide may be derived from an antibody directed against the target molecule. Specifically, this type of binding peptide is a single chain antibody (scFv) or the peptide comprises at least a complementarity-determining region 3 (CDR3) of an antibody. It is to be understood that it is not necessary for the application of the invention that the nature of the target molecule is known. Targeting ligands can be selected from combinatorial peptide libraries on the basis of differential binding to molecules expressed on the surface of different cell types. Useful combinatorial peptide libraries for the invention include those in which a large variety of peptides is displayed on the surface of filamentous bacteriophages. In this respect, libraries displaying scFv variants are particularly useful. Moreover, methods which increase the combinatorial diversity of the libraries make the number of targeting ligands that can be generated for use in the invention almost limitless. Such methods include PCR-based random mutagenesis techniques. It will be clear from the above that the invention is in no way limited by the nature of the target cell population, the target molecule on the cell surface, or the binding ligand. In an even more preferred embodiment, the knobless fiber has an enhanced thermostability. To this end, teachings on structural design of trimeric coiled-coils are at hand (Harbury et al., 1993 and 1994). Such improved fibers find applications where more complex ligands than short peptides are added.

It is to be understood that the methods and means of the present invention to develop truly targeted adenoviruses apply to any adenovirus, regardless the host range or serotype of the genome used to derive the truly targeted adenovirus, and regardless of its ability or inability to replicate or to replicate under certain conditions (conditional replication). The following examples are provided by way of illustrating the invention.

### EXAMPLES

### Example 1

Construction of Recombinant Plasmids and Adenoviral Vectors

The knobless Ad5 fiber genes TSC and TSFLC were constructed using PCR techniques. To construct TSC, Ad5 fiber sequences 31042-32250 were amplified using primers T-for (5′-CTA ATA CGA CTC ACT ATA GGC TCG Agc cac cAT GAA GCG CGC AAG ACC GTC-3′ (SEQ ID NO:5)) and CS-rev (5′-CAT CTC CGG AAC CGG TCC ACA AAG TTA GCT TAT C-3′ (SEQ ID NO:6)). T-for (SEQ ID NO:5) contains a XhoI-site (underlined) and sequences fulfilling the Kozak consensus (small case) upstream of the Ad5 fiber coding region (nt 31042-31061). CS-rev (SEQ ID NO:6) contains the antisense sequences of MoMLV envelope glycoprotein nt 7316-7319 (all MoMLV nt numbers are according to Shinnick et al., 1981), three codons for Gly-Ser-Gly (bold), and Ad5 fiber nt 32230-32250. This PCR yields the 1253 nt product XhoI-TS-GSG.

The MoMLV p15E helix domain was amplified using primers TC-for (5′-GTG GAC CGG TTC CGG AGA TGA TCT CAG GGA GGT TGA-3′ (SEQ ID NO:7)) and C-rev (5′-GCT AGG ATC CTC CAC CTC CGG AAC CTC CCC CTC CTT CTT TTA GAA ATA AC-3′ (SEQ ID NO:8)). TC-for (SEQ ID NO:7) contains Ad5 fiber nt 32244-32250, codons for Gly-Ser-Gly (bold) and MoMLV envelope glycoprotein nt 7316-7335. Antisense primer C-rev (SEQ ID NO:8) contains two sets of Gly₄-Ser linkers (bold) including a restriction site for BamHI (underlined) and MoMLV envelope glycoprotein nt 7414-7435 (the first two Gly residues of the flexible linker are from MoMLV coding region). This PCR results in the 164 nt fragment GSG-C-(GS)₂. Next, the XhoI-TS-GSG and GSG-C-(G₄S)₂ PCR products were mixed and amplified in a reassembly PCR with sense primer T-for (see SEQ ID NO:5 above) and antisense primer XFL-rev (5′-GCT CTA GAG CTA GGA TCC TCC ACC TCC-3′ (SEQ ID NO:9)), containing a XbaI site (bold) downstream from the C-terminal (G₄S)₂ linker (underlined). The final 1401 nt PCR product TSC links the Ad5 fiber tail and shaft regions via a GSG linker to the MoMLV helix domain and carries a C-terminal (G₄S)₂ linker with a unique BamHI cloning site for ligand addition.

To construct TSFLC, Ad5 fiber sequences 31042-32250 were amplified using primers T-for (see SEQ ID NO:5 above) and FLS-rev (5′-GCT ATC CTC CGG AAC CGC CTC CAC CGG TCC ACA AAG TTA GCT TAT C-3′ (SEQ ID NO:10)). FLS-rev (SEQ ID NO: 10) contains the antisense sequences of Gly₄-Ser-Gly₂ (bold) including a BspEI site (underlined), and Ad5 fiber nt 32230-32250. This PCR yields the 1265 nt product XhoI-TS-FL. The MoMLV p15E helix domain was amplified using primers FLC-for (5′-GGT TCC GGA GGA GGA GGA TCA GGT GGT GGT GGA TCA GAT GAT CTC AGG GAG GTT GA-3′ (SEQ ID NO:11) and C-rev (see SEQ ID NO:8 above). FLC-for (SEQ ID NO:11) contains codons for Gly-Ser-(Gly₄Ser)₂ (bold) including a BspEI site (underline), and MoMLV envelope glycoprotein nt 7316-7335. This PCR results in a 184 nt fragment FL-C-(G₄S)₂. XhoI-TS-FL and FL-C-(G₄S)₂ were each (partially) digested with BspEI, mixed, ligated, and amplified using primers T-for (see SEQ ID NO:5 above) and XFL-rev (see SEQ ID NO:9 above). The final 1437 nt PCR product TSFLC links the Ad5 fiber tail and shaft regions via a (G₄S)₃ linker (SEQ ID NO:3) to the MoMLV helix domain and a carries a C-terminal (G₄S)₂ linker (SEQ ID NO:4) with a unique BamHI cloning site for ligand addition.

Both PCR products TSC and TSFLC were cloned into pcDNA3 vector (Invitrogen) using the XhoI and XbaI sites, yielding constructs pCMV-TSC and pCMV-TSFLC, respectively. The entire inserts in these constructs were sequenced to confirm the correct construction of the fusion genes. The BamHI site in the pcDNA3 polylinker of pCMV-TSCand pCMV-TSFLC was removed by digestion with KpnI and EcoRV followed by blunt religation, to make the BamHI site in their C-terminal flexible linker unique. pCMV-TSCmychis and pCMV-TSFLCmychis were made by removing the NotI-BamHI fragment comprising the LacZ gene from pcDNA3.1(−)/Myc-His/LacZ (Invitrogen) and replacing it with the NotI-Bam-HI fragment from pCMV-TSC or pCMV-TSFLC encompassing the complete knobless fiber ORF with C-terminal flexible linker. In the ORFs of pCMV-TSCmychis and pCMV-TSFLCmychis the C-terminal (G₄S)₂ linker (SEQ ID NO:4) is followed by the 29 aa sequence ELGTKLGPEQKLISEEDLNSAVDHHHHHH (SEQ ID NO: 12), where the Myc-epitope and 6His-tag are underlined.

To generate pCMVtpl-TSCmychis and pCMVtpl-TSFLCmychis, the Ad2 tripartite leader sequence was amplified from construct pMad5 (Toes et al., 1997; a gift from DrFallaux, Leiden University, the Netherlands) using primers X-TPL (5′-TGC TCT AGA CTC TCT TCC GCA TCG CTG-3′ (SEQ ID NO:13)), containing a XbaI site (underlined), and TPL-E (5′-CAG GAA TTC TTG CGA CTG TGA CTG GTT AG-3′ (SEQ ID NO:14)), with an EcoRI site (underlined). This PCR amplifies Ad2 tripartite leader sequences 1-201 (Logan and Shenk, 1984) adding a 5′ XbaI site. After digestion with XbaI (in X-TPL primer (SEQ ID NO:13)) and XhoI (TPL nt 172) this fragment was inserted into XbaI and XhoI digested pCMV-TSCmychis or pCMV-TSFLCmychis. This results in the insertion of the 172 nt Ad2 tripartite leader fragment 5 nt upstream of the knobless fiber gene ATG.

To clone all knobless fiber genes downstream of the Ad2 Major Late Promoter and tripartite leader, the MLPtpl fragment was amplified from pMad5 using primers N-MLP (5′-CTA AGA ATG CGG CCG CGA GCG GTG TTC CGC GGT C-3′ (SEQ ID NO:15)), containing a NotI site (underlined) and TPL-E (see SEQ ID NO: 14 above). The 485 nt PCR product was digested with NotI and EcoRI and inserted into the polylinker of pBluescript II SK(−) (Stratagene) upstream of a 1.5 kb EcoRI-HindIII fragment from pTet-Off (Clontech). The correct sequence of the cloned PCR product was confirmed. After deletion of the 1.0 kb EcoRI-BamHI pTet-Off fragment, the Xhol-PmeI inserts of pCMVtpl-TSCmychis and pCMVtpl-TSFLCmychis, containing the entire knobless fiber ORFs, were blunt-end ligated into this vector between the MLPtpl PCR fragment and the SV40 pA signal from pTet-Off. The resulting constructs were designated pBSMUtpl-TSCmychis and pBSMLPtpl-TSFLCmychis, respectively. The NotI-ClaI inserts from pBSMLPtpl-TSCmychis and pBSMLPtpl-TSFLCmychis were isolated and the ClaI sites were made blunt-end. These fragments were cloned into pAdTrack (He et al., 1998) digested with NotI and KpnI with the latter site made blunt-end, giving constructs pAciTrackMLP-TSCmychis and pAdTrackMLP-TSFLCmychis, respectively.

### Example 2

Adenovirus Vector Production

Recombinant adenoviruses expressing wild-type fibers were produced by homologous recombination in 293 cells (Graham et al., 1977). Adenovirus backbone plasmid pAdEasy-1 (He et al., 1998) was digested with PacI and co-transfected into 293 cells together with PacI/PmeI-digested pAdTrack, pAdTrackMLP-TSCmychis or pAdTrackMLP-TSFLCmychis (see above) in a 1:1 molar ratio and a total amount of 4 mg DNA per T25 culture flask by Lipofectamine PLUS (Life Technologies) method according to the manufacturer's guidelines. After 8 days culture, the cells were harvested and virus was released by multiple freeze-thaw steps. After reinfection of fresh 293 cells total virus stocks were prepared and further expanded on 293 cells using standard procedures.

Purified virus stocks were prepared by two rounds of CsCl banding and dialysis against 10 mM HEPES pH 7.4 with 10% glycerol and 1 mM MgCl. Virus stocks were stored at −80° C. until use. Identity of the virus stocks was confirmed by PCR analysis using primers FLC- for (SEQ ID NO: 11) and C-rev (SEQ ID NO:8) specific for the MoMLV p15E helix domain. Virus particle titers were determined by OD₂₆₀ measurement after lysis in PBS containing 1% SDS and 1 mM EDTA at 55° C. for 10 minutes. Functional titers in infectious units (IU) were determined by end-point dilution infection on 293.HissFv.rec cells and scoring EGFP-expressing cells 6 days after infection. Typical titers produced were 9×10¹¹−2×10¹² particles/ml (particle/IU ratios ranging from 20:1 to 1:2). The absence of replication-competent adenovirus was confirmed by infection of A549 cells (AATC No. ______) with approximately 10⁹ IU of recombinant virus.

Example 3

Analysis of Knobless Fiber Expression

Transient transfection assays on 911 cells (Fallaux et al., 1996) were performed using Lipofectamine PLUS reagent (Life Technologies) according to the manufacturer's instructions. Expression analysis by immunocytochemistry was performed at 24 hours after transfection. Cells were fixed to the culture dish with an ice cold 50%/50% vol mixture of methanol and acetone for 15 min and washed thee times with PBS. Next, fixed cells were incubated 1 hour at 37° C. with 10 ug/ml anti-Myc MoAb 9E10 (Chan et al., 1987) diluted in PBS/0.5% BSA. After three washes with PBS, incubation with RbaMIgG-AP conjugate (Dako) diluted 1:100 in PBS/0.5% BSA was performed for 1 hour at room temperature (RT). The dishes were washed five times with PBS and incubated for 10-20 min at RT with BCIP/NBT substrate (Dako). Finally, the cells were washed twice with PBS, shortly counterstained with nuclear fast red, and washed in PBS twice again. Expression of Myc-epitope containing proteins was evaluated by microscopic inspection.

Knobless fiber expression from recombinant adenovirus vectors was examined after infection of 293 cells at high MOI. When the cells were in full CPE and completely detached from the culture flask (at day 2), they were harvested and washed in PBS. Cell lysates were prepared in PBS by four freeze-thaw cycles and three times 10 seconds sonification. Samples for Western immunoblot analysis were prepared by adding 2 volumes of Laemmli loading buffer (62.5 mM Tris-HCL pH 6.8, 25% v/v glycerol, 2% w/v SDS, 5% v/v beta-mercaptoethanol, 0.01% w/v bromophenol blue) and 5 minutes heating at 95° C. (denaturing condition) or by adding 2 volumes of a modified loading buffer with a reduced amount of SDS (0.2% w/v) and lacking beta-mercaptoethanol, without heating (semi-native condition). Samples were separated on 7.5% SDS-PAGE gels in Tris/glycine-buffer and transferred to PVDF membrane (Sequiblot; Bio-Rad, Hercules, Calif.) in the same buffer with 15% v/v methanol. After 1.5 h blocking with 5% w/v lowfat dry milk (LFDM) in Tris Buffered Saline with 0.1% v/v Tween-20 (TBST) at RT, blots were incubated overnight at 4° C. with 5 ug/ml anti-Myc MoAb 9E10 or 5 ug/ml anti-fiber knob MoAb 1D6.14 (Douglas et al., 1996) in LFDM-TBST. Following extensive washing in TBST, the blots were incubated for 1 hour at RT with 1:3000 diluted RbaMIgG-HRPO conjugate (Dako) in LFDM-TBST. After further extensive washing in TBST, the blots were developed using Lumilight^{PLUS} chemiluminescence detection reagent (Boehringer Mannheim) as per manufacturer's instructions.

Wild-type and knobless fibers on intact adenovirus particles were analyzed by Western analysis under semi-native conditions as described above for cell lysates, starting from CsCl purified virus stocks in HEPES buffer with 10% glycerol.

### Example 4

Ni-NTA Purification of 6His-Tagged Proteins and Viruses

Total protein lysates were prepared from Ad infected 293 cells in full CPE as described above. The lysates were cleared by centrifugation. One volume of 50% Ni-NTA Superflow resin slurry (Qiagen, Hilden, Germany) was equilibrated in 10 volumes PBS with 300 mM NaCl for 45 minutes at 4° C., following which the Ni-NTA resin was resuspended in its original volume. A mixture was prepared containing 70% v/v cleared protein lysate and 20% v/v equilibrated 50% Ni-NTA slurry in PBS with 300 mM NaCl and 5 mM imidazol. After 1 hour incubation at 4° C., Ni-NTA resin was spun down and the unbound material was harvested for analysis. The Ni-NTA slurry was washed twice with 3 volumes PBS with 300 mM NaCl and 5 mM imidazol and once with 3 volumes PBS with 300 mM NaCl and 30 mM imidazol. The supernatant of the 30 mM imidazol step was kept for analysis. Finally, specifically bound material was eluted by incubation with 3 volumes PBS with 300 MM NaCl and 300 mM imidazol. The eluted proteins were concentrated using Ultrafree-0.5 centrifugal filters with Biomax-10 membrane (Millipore) according to the manufacturer's instructions.

Intact virus particles (10¹² particles) prepared by CsCl banding and stored in dialysis buffer as described above were mixed with 1/10 volume Ni-NTA beads equilibrated in dialysis buffer and incubated for 5 hours at 40° C. by end-over-end rotation. The Ni-NTA beads were sedimented by gravity on ice and unbound material was aspirated. The beads were washed twice with 9 volumes dialysis buffer containing 5 mM imidazol, twice with 9 volumes buffer with 50 mM imidazol and once with 9 volumes buffer containing 250 mM imidazol. After each buffer incubation, the beads were sedimented by gravity on ice. The unbound fraction, the first wash at 5 mM imidazol, the first 50 mM imidazol elution, and the 250 mM imidazol elution fraction were kept for analysis.

### Example 5

### Virus DNA Analysis

Viral DNA was isolated from particles by 1 hour incubation at 37° C. in 0.6% SDS, 10 mM EDTA, 50 mg/ml proteinase K, followed by phenol extraction and purification over tip-20 columns (Qiagen) according to the instructions of the manufacturer. The DNA was visualized by 0.5% agarose gel electrophoresis and ethidium bromide staining.

### Example 6

Antibody-Mediated Virus Capture Assay

Ninety-six-well Microlon 200 ELISA plates (Greiner) were coated with 10 microgram/ml RbaMIgG antibodies (Dako) in PBS for 2 hours at 37° C. After two washes with PBS, the plates were incubated for 1.5 hour at 37° C. with PBS/1% BSA as a negative control or with 1 microgram/ml anti-fiber knob MoAb 1D6.14 or anti-Myc MoAb 9E10 in PBS/1% BSA. After three washes with PBS, 10-fold limiting dilution titrations of CsCl-purified AdGFP, AdGFP-TSCmychis, or AdGFP-TSFLCmychis virus were made in triplicate in DMEM/F12 medium (Life Technologies) with 2% v/v fetal bovine serum (FBS) starting from 5.108 particles per well and allowed to bind for 1 hour at 37° C. Unbound virus was vigorously removed by five washes with PBS. Next, 105 293.HissFv.rec cells (Douglas et al., 1999) in 100 microliter DMEM/F12 medium with 10% FBS were seeded into each well. After overnight culture at 37° C., GFP expression was evaluated by fluorescence microscopy and wells exhibiting GFP fluorescent cells in a linear range were selected. For wells coated with ID6.14 MoAb these were the wells loaded with 5.10⁶ particles, for negative control wells and wells coated with 9E10 MoAb the wells loaded with 5.1 0 particles were selected. Cells from selected wells were harvested by trypsinization, washed in PBS, fixated in PBS with 2.5% formaldehyde and analyzed for GFP fluorescence on a FACScan (Becton Dickinson) according to standard procedures. The titer of functional virus bound to the plates was calculated according to the following equation: percent GFP-expressing cells determined by FACS analysis x number of cells seeded x virus dilution. Titers of the viruses bound to 9E10 or negative control plates are expressed as percentages +/− SD relative to the binding to ID6.14.

### Example 7

Targeted Virus Infection Assay

293.HissFv.rec cells were seeded 1.10(5) cells per well in 24-well tissue culture plates 24 hours prior to infection. Virus was serially diluted in DMEM/F12 with 1% FBS with or without 50 ug/ml 1D6.14 anti-fiber knob MoAb and/or 20 ug/ml anti-Myc 9E10 MoAb, and incubated at RT for 30 minutes. Next, culture medium was removed from the cells and replaced with 200 microliter diluted virus to infect the cells at an MOI of 100 particles/cell for 30 minutes at RT. After this incubation, the medium was replaced by DMEM/F12 with 10% FBS and the cells were cultured 24-hours at 37° C. Finally, cells were harvested and single cells were analyzed for EGFP expression on a FACScan according to standard procedures.

### Example 8

Construction of a Knobless Virus Displaying a Single Chain Antibody Against a Widely Expressed Cell Surface Antigen

An adenoviral vector having a retroviral trimerization domain instead of its natural carboxy terminal trimerization domain and a single chain antibody sequence instead of its natural knob domain is constructed as follows. A phagemid clone of phage Fab antibody #25 (European Patent Appln. No. 98201693) was developed against the third predicted extracellular domain of hCAT1, a cationic amino acid transporter. The sequence of this antibody is used to construct a single chain sequence where the light and heavy chains were fused together by an amino acid linker introduced using PCR. This sequence is introduced into the vectors pCMV-TSCmychis or pCMV-TSFLCmychis, encoding a knobless monomeric fiber sequence with the MuLV envelope trimerization domain (TSC or TSFLC) as described in Example 1. This construct is then used to produce an adenoviral vector resulting in an adenovirus displaying a single chain antibody against hCAT1.

### Example 9

Universal Method to Construct TSFLC Knobless Fiber Derivatives with Targeting Specificity

First, a recombinant dsDNA fragment is constructed which comprises from 5′ to 3′ the following elements: a BamHI-site (GGATCC; encoding amino acids Gly and Ser), a DNA sequence coding for the binding ligand with or without a 6-His peptide placed in frame with the Gly and Ser residues of the BamHI-site, at least one stop codon placed in frame with the 5′ sequence and a polyadenylation signal, where it is preferred that the stop codon and polyadenylation signal overlap, where it is further preferred that they are of the sequence 5′-TAATAAA-3′, an NdeI-site (CATATG), and an XbaI-site (TCTAGA). The recombinant dsDNA fragment is made using standard molecular biology methods known in the art including, but not limited to, hybridization of overlapping synthetic oligonucleotides with or without polymerase treatment, PCR on a ligand-encoding template using primers comprising the flanking restriction sites and signal elements, and the like. Next, the recombinant dsDNA fragment is digested with BamHI and XbaI. The construct pCMV-TSFLC with removed pcDNA3 BamHI polylinker site (see Example 1) is also digested with BamHI and XbaI. The BamHI/XbaI-digested dsDNA fragment comprising the ligand encoding sequence is inserted, which places the binding ligand encoding sequence in frame with the knobless fiber ORF. The resulting construct pCMV-TSFLC-ligand may be used for analysis of the TSFLC-ligand protein in transient expression assays or for insertion into the adenovirus genome (see Example 10).

### Example 10

Method to Construct Adenoviruses in which the Endogenous Fiber Gene is Replaced with a Knobless Fiber Gene According to the Invention

First, the universal acceptor plasmid pBR.Ad.Bam-RITRdeltaFib-P for insertion of knobless fiber genes at the endogenous genomic locale of the fiber gene was made as follows. The 12 kb BamHI fragment from pAdEasy-1 (He et al., 1998) was sub-cloned into a pBR322-derivative that lacks the NdeI site. In the resulting construct pBR.Ad.Bam-RITR-P the NdeI site in the fiber ORF and the AvrII site near the 3′ ITR are both unique. In addition, we used the construct pBR.Ad.Bam-RITR. This construct carries the complete right-hand part of the wild-type Ad5 genome starting from the BamHI site at position 21562, cloned into the same NdeI-deficient pBR322 derivative as above. pBR.Ad.Bam-RITR was digested with NdeI and partially with Sse83871, to delete the fragment from Ad nt 31088 to Ad nt 33288. The deleted fragment was replaced by the PCR product NNdeltaFS that covers the Ad5 sequence from 7 nt down-stream from the fiber ORF until the Sse83871 site, and has 5′ added NdeI and NsiI sites. NNdeltaFS was made using primers 5′-CGA CAT ATG TAG ATG CAT TAG TTT GTG TTA TGT TTC AAC GTG-3′ (SEQ ID NO:16) (with NdeI and NsiI sites) and 5′-CCT CTG GAG ACG GTA CAA C-3′ (SEQ ID NO: 17), using wild-type Ad5 DNA as template, and subsequent digestion with NdeI and Sse83871. The resulting construct pBR.Ad.Bam-RITRdeltaFib carries a unique NsiI site in place of most of the fiber ORF (approximately 1.7 kb deleted). Next, the 4.3 kb Ndel-AvrII fragment from pBR.Ad.Bam-RITR-P was replaced by the 2.6 kb NdeI-AvrII fragment from pBR.Ad.Bam-RITRdeltaFib. This creates pBR.Ad.Bam-RITRdeltaFib-P, in which the fiber gene NdeI site is unique. As a second step, the knobless fiber ORF from pCMV-TSFLC-ligand is excised by NdeI digestion and this fragment is inserted into NdeI-digested pBR.Ad.Bam-RITRdeltaFib-P. Recombinants with an insert in the correct orientation (called pBR.Ad.Bam-RITR-TSFLC-ligand-P) carry the TSFLC-ligand ORF in place of the native fiber gene, and with its expression hence regulated in the proper genomic context. Finally, PacI-linearized pBR.Ad.Bam-RITR-TSFLC-ligand-P is allowed to recombine with SpeI-linearized pAdEasy-1 in E. coli BJ5183 cells, essentially as described by Chartier et al. (1996), to create pAdEasy-TSFLC-ligand.

pAdEasy-TSFLC-ligand can be used together with a plasmid construct that complements left-hand adenovirus sequences lacking in pAdEasy-TSFLC-ligand, such as a pShuttle, pShuttle-CMV, pAdTrack or pAdTrack-CMV derivative with or without a foreign gene insert of choice or adenovirus El region with or without mutations rendering the resulting virus replication-defective, replication-competent, or conditionally-replicating, to construct full-length truly targeted recombinant adenovirus genomes, by homologous recombination in E. coli BJ5183 cells as described by He et al. (1998). Truly targeted recombinant adenovirus is produced by transfecting the PacI-linearized full-length adenovirus genome from the resulting recombination product into adenovirus packaging cells known in the art, according to the method described by He et al. (1998).

### REFERENCES

Amberg N., Mei Y. and Wadell G., 1997. Fiber genes of adenoviruses with tropism for the eye and the genital tract. Virology 227:239-244.

Bergelson, J. M., Cunningham, J. A., Droguett, G., Kurt-Jones, E. A., Krithivas, A., Hong, J. S., Horwitz, M. S., Crowell, R. L. and Finberg, R. W. (1997). Isolation of a common receptor for coxsackie B virus and adenoviruses 2 and 5. Science 275:1320-1323.

Blaese, M., Blankenstein, T., Brenner, M., Cohen-Hagenauer, O., Gansbacher, B., Russel, S., Sorrentino, B. and Velu, T. (1995). Cancer Gene Ther. 2:291-297.

Bout A. (1997). Gene therapy, p. 167-182. In: D. J. A. Crommelin and R. D. Sindelar (ed.), Pharmaceutical Biotechnology, Harwood Academic Publishers.

Bout, A. (1996). Prospects for human gene therapy. Eur. J. Drug Met. and Pharma. 2, 175-179.

Brody, S. L. and Crystal, R. G. (1994). Adenovirus mediated in vivo gene transfer. Ann. N.Y. Acad. Sci. 716:90-101.

Bullougli, P. A., Hughson, F. M., Skehel, J. J., Wiley D. C. (1994). Structure of influenza haemagglutinin at the pH of membrane fusion. Nature 371:19-20.

Carter, A. J., Laird, J. R., Farb, A., Kufs, W., Wortham, D. C. and Virnani, R. (1994). Morphologic characteristics of lesion formation and time course of smooth muscle cell proliferation in a porcine proliferative restenosis model. J. Am. Coll. Cardiol. 24:1398-1405.

Chambers P., Pringle C. R., Easton A. J. (1990). Heptad repeat sequences are located adjacent to hydrophobic regions in several types of virus fusion glycoproteins. J. Gen. Virol. 71:3075-3080.

Chan S., Gabra H., Hill F., Evan G., Sikora K. (1987). A novel tumour marker related to the c-myc oncogene product. Mol. Cell Probes 1:73-82.

Chan, D. C., Fass, D., Berger, J. M., Kim, P. S. (1997). Core structure of gp41 from the HIV envelope glycoprotein. Cell 89:263.

Chartier C., Degryse E., Gantzer M., Dieterle A., Pavirani A., Melitali M. (1996). Efficient generation of recombinant adenovirus vectors by homologous recombination in Escherichia coli. J. Virol. 70: 4805-4810.

Chroboczek J., Ruigrok R. W. H., and Cusack S., 1995. Adenovirus fiber, p. 163 In: W. Doerfler and P. Bolun (ed.), The molecular repertoire of adenoviruses, 1. Springer-Verlag, Berlin.

Cohen C. and Parry D. A. (1990). Alpha-helical coiled coils and bundles: how to design an alpha-helical protein. Proteins 7: 1

Defer C., Belin M., Caillet-Boudin M. and Boulanger P., 1990. Human adenovirus-host cell interactions; comparative study with members of subgroup B and C. Journal of Virology 64(8): 3661-3673.

Douglas J. T., Miller C. R., Kim M., Dmitriev I., Mikheeva G., Krasnykh V., Curiel D. T. (1999). A system for the propagation of adenoviral vectors with genetically modified receptor specificities. Nature Biotech. 17:470-475.

Fallaux F. L, Kranenburg O., Cramer S. J., Houweling A., Van Ormondt H., Hoeben R. C., Van Der Eb A. J. (1996). Characterization of 911: a new helper cell line for the titration and propagation of early region 1-deleted adenoviral vectors. Hum. Gene Ther. 7:215-222.

Fallaux, F. J, Bout, A, van der Velde, I et al. New helper cells and matched E1-deleted adenovirus vectors prevent generation of replication competent adenoviruses. Human GeneTherapy, 9 (1998), p 1909-1917.

Fass D. and Kim P. S. (1995). Dissection of a retrovirus envelope protein reveals structural similarity to influenza hemagglutinin. Curr. Biol. 5:1377-1383.

Fass, D., Harrison, S. C., Kim, P. S. (1996). Retrovirus envelope domain at 1.7 angstrom resolution. Nat Struct Biol 3:465.

Francki, R. I. B., Fauquet, C. M., Knudson, D. L. and Brown, F. (1991). Classification and nomenclature of viruses. Fifth report of the international Committee on taxonomy of viruses. Arch. Virol. Suppl. 2:140-144.

Gall J., Kass-Eisler A., Leinwand L. and Falck-Pedersen E. (1996). Adenovirus type 5 and 7 capsid chimera: fiber replacement alters receptor tropism without affecting primary immune neutralization epitopes. Journal of Virology 70 (4):2116-2123.

Graham F. L., Smiley J., Russell W. C., Naim R. (1977). Characteristics of a human cell line transformed by DNA from human adenovirus type 5. J. Gen. Virol. 36:59-72.

Greber, U. F., Willets, M., Webster, P., and Helenius, A. (1993). Stepwise dismanteling of adenovirus 2 during entry into cells. Cell 75:477-486.

Harbury P. B., Zhang T., Kim P. S., Alber T. (1993). A switch between two-, three-, and four-stranded coiled coils in GCN4 leucine zipper mutants. Science 262:1401-1407.

Harbury P. B., Kim P. S., Alber T. (1994). Crystal structure of an isoleucine-zipper trimer. Nature 371:80-83.

He T. C., Zhou S., da Costa L. T., Yu J., Kinzler K. W., Vogelstein B. (1998). A simplified system for generating recombinant adenoviruses. Proc. Natl. Acad. Sci. USA 95:2509-2514.

Herz J. and Gerard R. D. (1993). Adenovirus-mediated transfer of low density lipoprotein receptor gene acutely accelerates cholesterol clearance in normal mice. Proc. Natl. Acad. Sci. USA 90(1993):2812-2816.

Hynes, R. O. (1992). Integrins: versatility, modulation and signalling in cell adhesion. Cell 69:11-25.

Hierholzer, J. C. (1992). Adenovirus in the immunocompromised host. Clin. Microbiol Rev. 5, 262-274.

Hierholzer, J. C., Wigand, R., Anderson, L. L, Adrian, T., and Gold, J. W. M. (1988). Adenoviruses from patients with AIDS: a plethora of serotypes and a description of five new scrotypes of subgenus D (types 43-47). J. Infect. Dis. 158, 804-813.

Hong, S. S., Karayan, L., Toumier, J., Curiel, D. T. and Boulanger, P. A. (1997). Adenovirus type 5 fiber knob binds to MHC class 1 (2 domain at the surface of human epithelial and B lymphoblastoid cells. EMBO J. 16:2294-2306.

Hong J. S. and Engler J. A. (1991). The amino terminus of the adenovirus fiber protein encodesthe nuclear localization signal. Virology 185:758-767.

Hsu, K. H., Lonberg-Holm, K., Alstein, B. and Crowell, R. L. (1988). A monoclonal antibody specific for the cellular receptor for the group B coxsackieviruses. J. Virol 62(5): 1647-1652.

Huard, J., Lochmuller, H., Acsadi, G., Jani, A., Massie, B. and Karpati, G. (1995). The route of administration is a major determinant of the transduction efficiency of rat tissues by adenoviral recombinants. Gene Ther. 2:107-115.

Imperiale, M. J., Akusjnarvi, G. and Leppard, K. N. (1995). Post-transcriptional control of adenovirus gene expression. In: The molecular repertoire of adenoviruses I. pp 139-171. W. Doerfier and P. Bohm (eds), Springer-Verlag Berlin Heidelberg.

Ishibashi, M. and Yasue, H. (1984). The adenoviruses, H. S. Ginsberg, ed., Plenum Press, Londen, New York. Chapter 12, 497-561.

Jaffe, E. A., Naclunan, R. L., Becker, C. G., Minick, C. R. (1973). Culture of endothelial cells derived from umbilical veins. Identification by morphologic and immunologic criteria. J. Clin. Invest. 52, 2745-2756.

Kass-Eisler, A., Falck-Pederson, E., Elfenbein, D. H., Alvira, M., Buttrick, P. M. and Leinwand, L. A. (1994). The impact of developmental stage, route of administration and the immune system on adenovirus-mediated gene transfer. Gene Ther. 1:395-402.

Khoo, S. H., Bailey, A. S., De Jong, J. C., and Mandal, B. K. (1995). Adenovirus infections in human immunodeficiency virus-positive patients: Clinical features and molecular epidemiology. J. Infect. Dis 172, 629-637.

Kidd, A. H., Chroboczek, J., Cusack, S., and Ruigrok, R. W. (1993). Adenovirus type 40 virions contain two distinct fibers. Virology 192, 73-84.

Krasnykh V. N., Milcheeva G. V., Douglas J. T. and Curiel D. T. (1996). Generation of recombinant adenovirus vectors with modified fibers for altering viral tropism. J. Virol. 70(10): 6839-6846.

Krasnykh V. N., Dmitriev I., Mikheeva G., Miller C. R., Belousova N. and Curiel D. T. (1998). Characterization of an adenovirus vector containing a heterologous peptide epitope in the HI loop of the fiber knob. J. Virol. 72(3): 1844-1852.

Law, L., Chillon, M., Bosch, A., Annentano, D., Welsh, M. J. and Davidson, B. L. (1998). Infection of primary CNS cells by different adenoviral serotypies: Searching for a more efficient vector. Abstract 1st Annual Meeting American Society of Gene Therapy, Seattle, Wash.

Leppard, K. N. (1997). E4 gene function in adenovirus, adenovirus vector and adeno-associated virus infections. J. Gen. Virol. 78: 2131-2138.

Lloyd Jones, D. M. and Bloch, K. D. (1996). The vascular biology of nitric oxide and its role in atherogenesis. Annu. Rev. Med. 47:365-375.

Logan J. and Shenk T. (1984). Adenovirus tripartite leader sequence enhances translation of mRNAs late after infection. Proc. Natl. Acad. Sci. USA 81: 3655-3659.

Mitraki A., Barge A., Chroboczek J., Andrieu J. P., Gagnon J., Ruigrok R. W. (1999). Unfolding studies of human adenovirus type 2 fibre trimers. Evidence for a stable domain. Eur. J. Biochem. 264:599-606.

Morgan, C., Rozenkrantz, H. S., and Mednis, B. (1969). Structure and development of virusesas observed in the electron microscope.X. Entry and uncoating of adenovirus. J. Virol 4, 777-796.

Novelli A. and Boulanger P. A. (1991). Deletion analysis of functional domains in baculovirus-expressed adenovirus type 2 fiber. Virology 185:365-376.

Roelvink, P. W., Kovesdi, I. and Wickham, T. J. (1996). Comparative analysis of adenovirus fiber-cell interaction: Adenovirus type 2 (Ad2) and Ad9 utilize the same cellular fiber receptor but use different binding stratagies for attachment. J. Virol. 70:7614-7621.

Roelvink, P. W., Lizonova, A., Lee, J. G. M., Li, Y., Bergelson, J. M., Finberg, R. W., Brough, D. E., Kovesdi, I. and Wickham, T. J. (1998. The comackie-adenovirus receptor protein can function as a cellular attachment protein for adenovirus serotypes from subgroups A, C, D, E, and F. J. Virol. 72:7909-7915.

Rogers, B. E., Douglas J. T., Ahlem, C., Buchsbaum, D. J., Frincke, J. and Curiel, D. T. (1997). Use of a novel cross-linking method to modify adenovirus tropism. Gene Ther. 4:1387-1392.

Santis G., Legrand V., Hong S. S., Davison E., Kirby I., Imler J. L., Finberg R. W., Bergelson J. M., Mehtali M., Boulanger P. (1999). Molecular determinants of adenovirus serotype 5 fibre binding to its cellular receptor CAR. J. Gen. Virol. 80:1519-1527.

Schulick, A. H., Vassalli, G., Dunn, P. F., Dong, G., Rade, J. J., Zarnarron, C. and Dichek, D. A. (1997). Established immunity precludes adenovirus-mediated gene transfer in rat carotid arteries.

Schnurr, D and Dondero, M. E. (1993). Two new candidate adenovirus serotypes. Intervirol. 36. 79-83.

Schwartz, R. S., Edwards, W. D., Huber, K. C., Antoniudes, L. C. Bailey, K. R., Carnrud, A. R., Jorgenson, M. A. and Holmes, D. R. Jr. (1993). Coronary restenosis: Prospects for solution andnew perspectives from a porcine model. Mayo Clin. Proc. 68:54-62.

Shi, Y., Pieniek, M., Fard, A., O=Brien, J., Mannion, J. D. and Zalewski, A. (1996). Adventitial remodelling after coronary arterial injury. Circulation 93:340-348.

Shabram, P. W., Giroux, D. D., Goudreau, A. M., Gregory, R. J., Hom, N. T., Huyghe, B. G., Liu, X., Nunnally, M. H., Sugarman, B. J. and Suffipto, S. (1997). Analytical anion-exchange HPLC of recombinant type-5 adenoviral particles. Hum. Gene Ther. 8(4):453-465.

Shinnick T. M., Lemer R. A., Sutcliffe J. G. (1981). Nucleotide sequence of Moloney murine leukaemia virus. Nature 293:543-548.

Signas, G., Akusjarvi, G., and Petterson, U. (1985). Adenovirus 3 fiberpolypeptide gene: Complications for the structure of the fiber protein. J. Virol. 53, 672-678.

Stevenson S. C., Rollence M., Wlute B., Weaver L. and McClelland A., (1995). Human adenovirus serotypes 3 and 5 bind to two different cellular receptors via the fiber head domain. J. Virol 69(5):2850-2857.

Stevenson S. C., Rollence M., Marshall-Neff J. and McClelland A. (1997). Selective targeting of human cells by a chimaeric adenovirus vector containing a modified fiber protein. J. Virology 71(6):4782-4790.

Stouten, P. W. F., Sander, C., Ruigrok, R. W. H., and Cusack, S. (1992). New triple helical model for the shaft of the adenovirus fiber. J. Mol. Biol. 226, 1073-1084.

Svensson, V. and Persson, R. (1984). Entry of adenovirus 2 into Hela cells. J. Virol. 51, 687-694.

Toes et al. (1997) Proc. Natl. Acad. Sci. USA 94:14660-14665.

Van der Vliet, P. C. (1995). Adenovirus DNA replication In: W. Doerfier and P. Bohm (eds.), The molecular repertoire of adenoviruses II. pringer-Verlag, Berlin.

Van Raaij M. J., Mitraki A., Lavigne G., Cusack S. (1999). A triple beta-spiral in the adenovirus fibre shaft reveals a new structural motif for a fibrous protein. Nature 401: 935-938.

Varga, M. J., Weibull, C., and Everitt, E. (1991). Infectious entry pathway of adenovirus type 2. J. Virol 65, 6061-6070.

Varenne, O., Pislaru, S., Gillijns, H., Van Pelt, N., Gerard, R. D., Zoldhelyi, P., Van de Werf, F., Collen, D. and Janssens, S. P. (1998). Local adenovirus-mediated transfer of human endothelial nitric oxide synthetase reduces luminal narrowing after coronary angioplasty in pigs. Circulation 98: 919-926.

Wadell, G. (1984). Molecular Epidemiology of human adenoviruses. Curr. Top. Microbiol. Immunol. 110, 191-220.

Wickham T. J., Carrion M. E. and Kovesdi I., 1995. Targeting of adenovirus penton base to new receptors through replacement of its RGD motif with other receptor-specific peptide motifs. Gene Therapy 2:750-756.

Wickham T. J., Segal, D. M., Roelvink, P. W., Carrion M. E., Lizonova, A., Lee, G-M., and Kovesdi, I. (1996). Targeted adenovirus gene transfer to endothelial and smooth muscle cells by using bispecific antibodies. J. Virol. 70 (10), 6831-6838.

Wickham, T. J., Mathias, P., Cherish, D. A., and Nemerow, G. R. (1993). Integrins avb3 and avb5 promote adenovirus internalization but not virus attachment. Cell 73, 309-319.

Wijnberg, M. J., Quax, P. H. A., Nieuwenbroek, N. M. E., Verheijen, J. H. (1997). The migration of human smooth muscle cells in vitro is mediated by plasminogen activation and can be inhibited by al-pha(2)-macro globulin receptor associated protein. Thromb. and Haemostas. 78, 880-886.

Wild, C., Dubay, J. W., Greemell, T., Baird, T. Jr, Oas, T. G., McDanal, C., Hunter, E., Matthews, T. (1994). Propensity for a leucine zipper-like domain of human immunodeficiency virus type 1 gp41 to form oligomers correlates with a role in virus-induced fusion rather than assembly of the glycoprotein complex. Proc Natl. Acad. Sci. USA, 1994. 91:12676.

Wold, W. S., Tollefson, A. E. and Hermiston, T. W. (1995). E3 transcription unit of adenovirus. In: W. Doerfler and P. Bohm (eds.), The molecular repertoire of adenoviruses I. Springer-Verlag, Berlin.

Zabner, J., Annentano, D., Chillon, M., Wadsworth, S. C. and Welsh, M. J. (1998). Type 17 fiber enhances gene transfer. Abstract 1 st Annual Meeting American Society of Gene Therapy, Seattle, Wash.

Having thus described the invention, what is claimed is:

1. An adenoviral vector comprising at least one normative amino acid sequence, wherein said normative amino acid sequence replaces the knob domain of the adenovirus fiber protein and provides said adenoviral vector with desired cell type specificity.

2. The adenoviral vector of claim 1, wherein said normative amino acid sequence is a binding ligand for binding to a specific cell type.

3. The adenoviral vector of claim 2, wherein said binding ligand for binding to a specific cell type is coupled to said adenoviral vector via a flexible linker peptide.

4. The adenoviral vector of claim 3, wherein said binding ligand for binding to a specific cell type comprises a trimerization domain.

5. The adenoviral vector of claim 4, wherein said trimerization domain is derived from a viral membrane fusion protein.

6. The adenoviral vector of claim 5, wherein said trimerization domain is derived from a retroviral envelope glycoprotein.

7. The adenoviral vector of claim 6, wherein said trimerization domain is derived from one of a Moloney Murine Leukemia Virus and a Rous Sarcoma virus.

8. The adenoviral vector of claim 7, wherein said binding ligand for binding to a specific cell type comprises a Myc-epitope.

9. The adenoviral vector of claim 8, wherein said binding ligand for binding to a specific cell type comprises a Myc-epitope and a 6His-peptide.

10. The adenoviral vector of claim 9, wherein said binding ligand for binding to a specific cell type is a monoclonal antibody, or a derivative thereof, directed against the extracellular domain of a cationic amino acid transporter protein.

11. The adenoviral vector of claim 10, wherein said cationic amino acid transporter protein is hCAT 1.

12. The adenoviral vector of claim 2, wherein said binding ligand for binding to a specific cell type is a monoclonal antibody, or a derivative thereof, against an epithelial cell adhesion molecule.

13. The adenoviral vector of claim 12, wherein said epithelial cell adhesion molecule is the 17-1A antigen.

14. The adenoviral vector of claim 1, wherein said normative amino acid has more than 25 amino acids.

15. A method of improving the recognition by an adenoviral vector for a specific cell, said method comprising:
contacting said specific cell with the adenoviral vector of any claim 1.

16. A cell, infected with an adenoviral vector of claim 1.

17. A tissue comprising the cell of claim 16.

18. A non-human animal comprising the cell of claim 16.

19. A method of producing a library of adenoviral vectors of claim 1 with desired cell type specificity for use in functional genomics applications, said method comprising:
assembling said adenoviral vectors in a cell line capable of doing so, in which the normative amino acid sequence encoded by a nucleic acid is expressed in said cell.

20. An adenoviral vector comprising a binding ligand for binding to a specific cell type, said binding ligand replacing the knob domain of an adenovirus fiber protein and providing said adenoviral vector with desired cell type specificity, said binding ligand comprising a viral membrane fusion protein trimerization domain.

21. The adenoviral vector of claim 20, wherein said trimerization domain is of one of a retroviral envelope glycoprotein, Moloney Murine Leukemia Virus, and Rous Sarcoma virus origin.

22. The adenoviral vector of claim 20, wherein said binding ligand for binding to a specific cell type comprises a Myc-epitope.

23. The adenoviral vector of claim 20, wherein said binding ligand for binding to a specific cell type comprises a Myc-epitope and a 6His-peptide.

24. The adenoviral vector of claim 20, wherein said binding ligand for binding to a specific cell type is a monoclonal antibody directed against the extracellular domain of a cationic amino acid transporter protein.

25. The adenoviral vector of claim 24, wherein said cationic amino acid transporter protein is hCAT 1.

26. The adenoviral vector of claim 20, wherein said binding ligand for binding to a specific cell type is a monoclonal antibody directed against an epithelial cell adhesion molecule.

27. The adenoviral vector of claim 26, wherein said epithelial cell adhesion molecule is the 17-1A antigen.


SEQUENCE LISTING (17 sequences)

SEQ ID NO: 1 - protein, 6 aa, Adenovirus
  MISC_FEATURE (1)..(6): conserved region in tail of Adenovirus serotypes
FNPVYP

SEQ ID NO: 2 - protein, 4 aa, Adenovirus serotype 5
  MISC_FEATURE (1)..(4): highly conserved motif that delineates start of fiber knob
TLWT

SEQ ID NO: 3 - protein, 15 aa, Artificial Sequence
  linker for single chain antibodies
GGGGSGGGGSGGGGS

SEQ ID NO: 4 - protein, 10 aa, Artificial Sequence
  flexible linker with a BamHI restriction site
GGGGSGGGGS

SEQ ID NO: 5 - DNA, 51 bp, Artificial Sequence
  primer T-for
ctaatacgactcactataggctcgagccaccatgaagcgcgcaagaccgtc

SEQ ID NO: 6 - DNA, 34 bp, Artificial Sequence
  primer CS-rev
catctccggaaccggtccacaaagttagcttatc

SEQ ID NO: 7 - DNA, 36 bp, Artificial Sequence
  primer TC-for
gtggaccggttccggagatgatctcagggaggttga

SEQ ID NO: 8 - DNA, 50 bp, Artificial Sequence
  primer C-rev
gctaggatcctccacctccggaacctccccctccttcttttagaaataac

SEQ ID NO: 9 - DNA, 27 bp, Artificial Sequence
  primer XFL-rev
gctctagagctaggatcctccacctcc

SEQ ID NO: 10 - DNA, 46 bp, Artificial Sequence
  primer FLS-rev
gctatcctccggaaccgcctccaccggtccacaaagttagcttatc

SEQ ID NO: 11 - DNA, 56 bp, Artificial Sequence
  primer FLC-for
ggttccggaggaggaggatcaggtggtggtggatcagatgatctcagggaggttga

SEQ ID NO: 12 - protein, 29 aa, Artificial Sequence
  portion of the open reading frame of p-CMV-TSCmychis and PCMV-TSFLSmychis
ELGTKLGPEQKLISEEDLNSAVDHHHHHH

SEQ ID NO: 13 - DNA, 27 bp, Artificial Sequence
  primer X-TPL
tgctctagactctcttccgcatcgctg

SEQ ID NO: 14 - DNA, 29 bp, Artificial Sequence
  primer TPL-E
caggaattcttgcgactgtgactggttag

SEQ ID NO: 15 - DNA, 34 bp, Artificial Sequence
  primer N-MLP
ctaagaatgcggccgcgagcggtgttccgcggtc

SEQ ID NO: 16 - DNA, 42 bp, Artificial Sequence
  primer
cgacatatgtagatgcattagtttgtgttatgtttcaacgtg

SEQ ID NO: 17 - DNA, 19 bp, Artificial Sequence
  primer
cctctggagacggtacaac